(19) United States
(12) Patent Application Publication
(10) Pub. No.: US 2010/0261767 A1
(43) Pub. Date: Oct. 14, 2010

(54) OXADIAZOLE DERIVATIVES FOR USE AS S1P1 AGONISTS IN THE TREATMENT OF AUTOIMMUNE AND INFLAMMATORY DISORDERS

(76) Inventors: Jag Paul Heer, Essex (GB); Thomas Daniel Heightman, Essex (GB); John Skidmore, Essex (GB)

Correspondence Address: GlaxoSmithKline;GLOBAL PATENTS -US, UW2220, P. O. BOX 1539, KING OF PRUSSIA, PA, 19406-0939, US

(21) Appl. No.: 12/747,183

(22) PCT Filed: Dec. 19, 2008
(86) PCT No.: PCT/EP08/67971

§ 371 Date: Jun. 10, 2010

(30) Foreign Application Priority Data
Dec. 21, 2007 (GB) 0725104.4

(51) Int. Cl. A61K 31/4245; C07D 271/06; A61P 43/00

(52) U.S. Cl. 514/364; 548/131

(57) ABSTRACT

The present invention relates to novel oxadiazole derivatives having pharmacological activity, processes for their preparation, pharmaceutical compositions containing them and their use in the treatment of various disorders.

The present invention relates to novel oxadiazole derivatives having pharmacological activity, processes for their preparation, pharmaceutical compositions containing them and their use in the treatment of various disorders.

Sphingosine 1-phosphate (S1P) is a bioactive lipid mediator formed by the phosphorylation of sphingosine by sphingosine kinases and is found in high levels in the blood. It is produced and secreted by a number of cell types, including those of hematopoietic origin such as platelets and mast cells (Okamoto et al 1998 J Biol Chem 273(42):27104; Sanchez and Hla 2004, J Cell Biochem 92:913). It has a wide range of biological actions, including regulation of cell proliferation, differentiation, motility, vascularisation, and activation of inflammatory cells and platelets (Pyne and Pyne 2000, Biochem J. 349: 385). Five subtypes of S1P responsive receptor have been described, S1P1 (Edg-1), S1P2 (Edg-5), S1P3 (Edg-3), S1P4 (Edg-6), and S1P5 (Edg-8), forming part of the G-protein coupled endothelial differentiation gene family of receptors (Chun et al 2002 Pharmacological Reviews 54:265, Sanchez and Hla 2004 J Cellular Biochemistry, 92:913). These 5 receptors show differential mRNA expression, with S1P1-3 being widely expressed, S1P4 expressed on lymphoid and hematopoietic tissues and S1P5 primarily in brain and to a lower degree in spleen. They signal via different subsets of G proteins to promote a variety of biological responses (Kluk and Hla 2002 Biochem et Biophysica Acta 1582:72, Sanchez and Hla 2004, J Cellular Biochem 92:913).

Proposed roles for the S1P1 receptor include lymphocyte trafficking, cytokine induction/suppression and effects on endothelial cells (Rosen and Goetzl 2005 Nat Rev Immunol. 5:560). Agonists of the S1P1 receptor have been used in a number of autoimmune and transplantation animal models, including Experimental Autoimmune Encephalomyelitis (EAE) models of MS, to reduce the severity of the induced disease (Brinkman et al 2003 JBC 277:21453; Fujino et al 2003 J Pharmacol Exp Ther 305:70; Webb et al 2004 J Neuroimmunol 153:108; Rausch et al 2004 J Magn Reson Imaging 20:16). This activity is reported to be mediated by the effect of S1P1 agonists on lymphocyte circulation through the lymph system. Treatment with S1P1 agonists results in the sequestration of lymphocytes within secondary lymphoid organs such as the lymph nodes, inducing a reversible peripheral lymphopenia in animal models (Chiba et al 1998, J Immunology 160:5037, Forrest et al 2004 J Pharmacol Exp Ther 309:758; Sanna et al 2004 JBC 279:13839). Published data on agonists suggests that compound treatment induces loss of the S1P1 receptor from the cell surface via internalisation (Graler and Goetzl 2004 FASEB J 18:551; Matloubian et al 2004 Nature 427:355; Jo et al 2005 Chem Biol 12:703) and it is this reduction of S1P1 receptor on immune cells which contributes to the reduction of movement of T cells from the lymph nodes back into the blood stream.

S1P1 gene deletion causes embryonic lethality. Experiments to examine the role of the S1P1 receptor in lymphocyte migration and trafficking have included the adoptive transfer of labelled S1P1 deficient T cells into irradiated wild type mice. These cells showed a reduced egress from secondary lymphoid organs (Matloubian et al 2004 Nature 427:355).

S1P1 has also been ascribed a role in endothelial cell junction modulation (Allende et al 2003 102:3665, Blood Singelton et al 2005 FASEB J 19:1646). With respect to this endothelial action, S1P1 agonists have been reported to have an effect on isolated lymph nodes which may be contributing to a role in modulating immune disorders. S1P1 agonists caused a closing of the endothelial stromal ‘gates’ of lymphatic sinuses which drain the lymph nodes and prevent lymphocyte egress (Wei wt al 2005, Nat. Immunology 6:1228).

The immunosuppressive compound FTY720 (JP11080026-A) has been shown to reduce circulating lymphocytes in animals and man, have disease modulating activity in animal models of immune disorders and reduce remission rates in relapsing remitting Multiple Sclerosis (Brinkman et al 2002 JBC 277:21453, Mandala et al 2002 Science 296:346, Fujino et al 2003 J Pharmacology and Experimental Therapeutics 305:45658, Brinkman et al 2004 American J Transplantation 4:1019, Webb et al 2004 J Neuroimmunology 153:108, Morris et al 2005 Eur J Immunol 35:3570, Chiba 2005 Pharmacology and Therapeutics 108:308, Kahan et al 2003, Transplantation 76:1079, Kappos et al 2006 New Eng J Medicine 335:1124). This compound is a prodrug that is phosphorylated in vivo by sphingosine kinases to give a molecule that has agonist activity at the S1P1, S1P3, S1P4 and S1P5 receptors. Clinical studies have demonstrated that treatment with FTY720 results in bradycardia in the first 24 hours of treatment (Kappos et al 2006 New Eng J Medicine 335:1124). The bradycardia is thought to be due to agonism at the S1P3 receptor, based on a number of cell based and animal experiments. These include the use of S1P3 knock-out animals which, unlike wild type mice, do not demonstrate bradycardia following FTY720 administration and the use of S1P1 selective compounds. (Hale et al 2004 Bioorganic & Medicinal Chemistry Letters 14:3501, Sanna et al 2004 JBC 279:13839, Koyrakh et al 2005 American J Transplantation 5:529)

Hence, there is a need for S1P1 receptor agonist compounds with selectivity over S1P3 which might be expected to show a reduced tendency to induce bradycardia.

The following patent applications describe oxadiazole derivatives as S1P1 agonists: WO03/105771, WO05/058848, WO06/047195, WO06/100633, WO06/115188, WO06/131336, WO07/024,922 and WO07/116,866.

The following patent applications describe tetrahydroisoquinolinyl-oxadiazole derivatives as S1P receptor agonists: WO06/064757, WO06/001463, WO04/113330.

A structurally novel class of compounds has now been found which provides agonists of the S1P1 receptor.

The present invention therefore provides compounds of formula (I) or a pharmaceutically acceptable salt thereof:

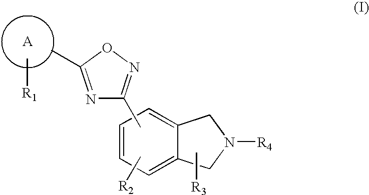

A is phenyl or a 5 or 6-membered heteroaryl ring;

R₁ is up to two substituents independently selected from halogen, C₍₁₋₃₎alkoxy, C₍₁₋₃₎fluoroalkyl, cyano, optionally substituted phenyl, C₍₁₋₃₎fluoroalkoxy, C₍₁₋₆₎alkyl and C₍₃₋₆₎cycloalkyl;

R₂ is hydrogen, halogen or C₁₋₄alkyl;

R₃ is hydrogen or C₁₋₃alkyl;

R₄ is hydrogen or (CH₂)₁₋₃CO₂H;

when R₂ or R₃ is C₁₋₃alkyl it may be optionally interrupted by O.

In one embodiment of the invention,

A is phenyl; and/or

R₁ is up to two substituents independently selected from chloro, isopropoxy, and cyano; and/or

R₂ is hydrogen; and/or

R₃ is hydrogen; and/or

R₄ is hydrogen, (CH₂)₂CO₂H or (CH₂)₃CO₂H.

In one embodiment of the invention,

A is phenyl;

R₁ is up to two substituents independently selected from chloro, isopropoxy, and cyano;

R₂ is hydrogen;

R₃ is hydrogen;

R₄ is hydrogen or (CH₂)₁₋₃CO₂H.

In one embodiment A is phenyl. In another embodiment A is 3,4-disubstituted phenyl.

In one embodiment R₁ is two substituents one of which is C₍₁₋₃₎alkoxy, the other selected from halogen or cyano. In another embodiment R₁ is two substituents, one of which is isopropoxy and the other is selected from chloro or cyano. In another embodiment R₁ is two substituents selected from chloro, isopropoxy and cyano. In another embodiment R₁ is chloro and isopropoxy. In a further embodiment R₁ is chloro at the 3-position and isopropoxy at the 4-position when A is phenyl. In another embodiment R₁ is isopropoxy and cyano. In a further embodiment R₁ is cyano at the 3-position and isopropoxy at the 4-position when A is phenyl.

In one embodiment R₂ is hydrogen.

In one embodiment R₃ is hydrogen.

In one embodiment R₄ is hydrogen or (CH₂)₁₋₃CO₂H. In another embodiment R₄ is hydrogen or (CH₂)₂₋₃CO₂H. In a further embodiment R₄ is hydrogen.

The term “alkyl” as a group or part of a group e.g. alkoxy or hydroxyalkyl refers to a straight or branched alkyl group in all isomeric forms. The term “C₍₁₋₆₎ alkyl” refers to an alkyl group, as defined above, containing at least 1, and at most 6 carbon atoms

Examples of such alkyl groups include methyl, ethyl, propyl, iso-propyl, n-butyl, iso-butyl, sec-butyl, or tert-butyl. Examples of such alkoxy groups include methoxy, ethoxy, propoxy, iso-propoxy, butoxy, iso-butoxy, sec-butoxy and tert-butoxy.

Suitable C₍₃₋₆₎cycloalkyl groups include cyclopropyl, cyclobutyl, cyclopentyl and cyclohexyl.

As used herein, the term “halogen” refers to fluorine (F), chlorine (Cl), bromine (Br), or iodine (I) and the term “halo” refers to the halogen: fluoro (—F), chloro (—Cl), bromo (—Br) and iodo (—I).

The term “heteroaryl” represents an unsaturated ring which comprises one or more heteroatoms selected from O, N or S. Examples of 5 or 6 membered heteroaryl rings include pyrrolyl, triazolyl, thiadiazolyl, tetrazolyl, imidazolyl, pyrazolyl, isothiazolyl, thiazolyl, isoxazolyl, oxazolyl, oxadiazolyl, furazanyl, furanyl, thienyl, pyridyl, pyrimidinyl, pyrazinyl, pyridazinyl and triazinyl.

In certain of the compounds of formula (I), dependent upon the nature of the substituent there are chiral carbon atoms and therefore compounds of formula (I) may exist as stereoisomers. The invention extends to all optical isomers such as stereoisomeric forms of the compounds of formula (I) including enantiomers, diastereoisomers and mixtures thereof, such as racemates. The different stereoisomeric forms may be separated or resolved one from the other by conventional methods or any given isomer may be obtained by conventional stereoselective or asymmetric syntheses.

Certain of the compounds herein can exist in various tautomeric forms and it is to be understood that the invention encompasses all such tautomeric forms.

It is understood that certain compounds of the invention contain both acidic and basic groups and may therefore exist as zwitterions at certain pH values.

Suitable compounds of the invention are:
5-[3-(2,3-Dihydro-1H-isoindol-4-yl)-1,2,4-oxadiazol-5-yl]-2-[(1-methylethyl)oxy]benzonitrile
4-(5-{3-Chloro-4-[(1-methylethyl)oxy]phenyl}-1,2,4-oxadiazol-3-yl)-2,3-dihydro-1H-isoindole
3-[4-(5-{3-Chloro-4-[(1-methylethyl)oxy]phenyl}-1,2,4-oxadiazol-3-yl)-1,3-dihydro-2H-isoindol-2-yl]propanoic acid
4-[4-(5-{3-Chloro-4-[(1-methylethyl)oxy]phenyl}-1,2,4-oxadiazol-3-yl)-1,3-dihydro-2H-isoindol-2-yl]butanoic acid
4-[4-(5-{3-cyano-4-[(1-methylethyl)oxy]phenyl}-1,2,4-oxadiazol-3-yl)-1,3-dihydro-2H-isoindol-2-yl]butanoic acid
   or pharmaceutically acceptable salts thereof.

Pharmaceutically acceptable derivatives of compounds of formula (I) include any pharmaceutically acceptable salt, ester or salt of such ester of a compound of formula (I) which, upon administration to the recipient is capable of providing (directly or indirectly) a compound of formula (I) or an active metabolite or residue thereof.

The compounds of formula (I) can form salts. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. Certain of the compounds of formula (I) may form acid addition salts with one or more equivalents of the acid. The present invention includes within its scope all possible stoichiometric and non-stoichiometric forms. Salts may also be prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropylamine, tris(hydroxymethyl)aminomethane (TRIS, trometamol) and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

Pharmaceutically acceptable acid addition salts may be prepared conventionally by reaction with the appropriate acid or acid derivative. Pharmaceutically acceptable salts with bases may be prepared conventionally by reaction with the appropriate inorganic or organic base.

The compounds of formula (I) may be prepared in crystalline or non-crystalline form, and, if crystalline, may optionally be hydrated or solvated. This invention includes within its scope stoichiometric hydrates or solvates as well as compounds containing variable amounts of water and/or solvent.

Included within the scope of the invention are all salts, solvates, hydrates, complexes, polymorphs, prodrugs, radiolabelled derivatives, stereoisomers and optical isomers of the compounds of formula (I).

The potencies and efficacies of the compounds of this invention for the S1P1 receptor can be determined by GTPγS assay performed on the human cloned receptor. Compounds of formula (I) have demonstrated agonist activity at the S1P1 receptor, using functional assays described herein.

Compounds of formula (I) and their pharmaceutically acceptable salts are therefore of use in the treatment of conditions or disorders which are mediated via the S1P1 receptor. In particular the compounds of formula (I) and their pharmaceutically acceptable salts are of use in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes (herein after referred to as the “Disorders of the Invention”).

Compounds of formula (I) and their pharmaceutically acceptable salts are therefore of use in the treatment of lupus erythematosis.

Compounds of formula (I) and their pharmaceutically acceptable salts are therefore of use in the treatment of psoriasis.

Compounds of formula (I) and their pharmaceutically acceptable salts are therefore of use in the treatment of multiple sclerosis.

It is to be understood that “treatment” as used herein includes prophylaxis as well as alleviation of established symptoms.

Thus the invention also provides compounds of formula (I) or pharmaceutically acceptable salts thereof, for use as therapeutic substances, in particular in the treatment of the conditions or disorders mediated via the S1P1 receptor. In particular the invention provides a compound of formula (I) or a pharmaceutically acceptable salt thereof for use as a therapeutic substance in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

Compounds of formula (I) and their pharmaceutically acceptable salts are of use as therapeutic substances in the treatment of lupus erythematosis.

Compounds of formula (I) and their pharmaceutically acceptable salts are of use as therapeutic substances in the treatment of psoriasis.

Compounds of formula (I) and their pharmaceutically acceptable salts are of use as therapeutic substances in the treatment of multiple sclerosis.

The invention further provides a method of treatment of conditions or disorders in mammals including humans which can be mediated via the S1P1 receptor, which comprises administering to the sufferer a therapeutically safe and effective amount of a compound of formula (I) or a pharmaceutically acceptable salt thereof. In particular the invention provides a method of treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes, which comprises administering to the sufferer a therapeutically safe and effective amount of a compound of formula (I) or a pharmaceutically acceptable salt thereof.

The invention provides a method of treatment of lupus erythematosis, which comprises administering to the sufferer a therapeutically safe and effective amount of a compound of formula (I) or a pharmaceutically acceptable salt thereof.

The invention provides a method of treatment of psoriasis, which comprises administering to the sufferer a therapeutically safe and effective amount of a compound of formula (I) or a pharmaceutically acceptable salt thereof.

The invention provides a method of treatment of multiple sclerosis, which comprises administering to the sufferer a therapeutically safe and effective amount of a compound of formula (I) or a pharmaceutically acceptable salt thereof.

In another aspect, the invention provides for the use of a compound of formula (I) or a pharmaceutically acceptable salt thereof in the manufacture of a medicament for use in the treatment of the conditions or disorders mediated via the S1P1 receptor.

In particular the invention provides a compound of formula (I) or a pharmaceutically acceptable salt thereof for use in the manufacture of a medicament for use in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

Compounds of formula (I) and their pharmaceutically acceptable salts are of use in the manufacture of a medicament for use in the treatment of lupus erythematosis.

Compounds of formula (I) and their pharmaceutically acceptable salts are of use in the manufacture of a medicament for use in the treatment of psoriasis.

Compounds of formula (I) and their pharmaceutically acceptable salts are of use in the manufacture of a medicament for use in the treatment of multiple sclerosis.

In order to use the compounds of formula (I) and pharmaceutically acceptable salts thereof in therapy, they will normally be formulated into a pharmaceutical composition in accordance with standard pharmaceutical practice. The present invention also provides a pharmaceutical composition, which comprises a compound of formula (I) or a pharmaceutically acceptable salt thereof, and a pharmaceutically acceptable carrier or excipient.

In a further aspect, the present invention provides a process for preparing a pharmaceutical composition, the process comprising mixing a compound of formula (I) or a pharmaceutically acceptable salt thereof and a pharmaceutically acceptable carrier or excipient.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents (e.g. pregelatinised maize starch, polyvinylpyrrolidone or hydroxypropyl methylcellulose); fillers (e.g. lactose, microcrystalline cellulose or calcium hydrogen phosphate); tabletting lubricants (e.g. magnesium stearate, talc or silica); disintegrants (e.g. potato starch or sodium starch glycollate); and acceptable wetting agents (e.g. sodium lauryl sulphate). The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents (e.g. sorbitol syrup, cellulose derivatives or hydrogenated edible fats), emulsifying agents (e.g. lecithin or acacia), non-aqueous vehicles (which may include edible oils e.g. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for oral administration may be suitably formulated to give controlled release of the active compound.

For parenteral administration, fluid unit dosage forms are prepared utilising a compound of the invention or pharmaceutically acceptable salts thereof and a sterile vehicle. Formulations for injection may be presented in unit dosage form e.g. in ampoules or in multi-dose, utilising a compound of the invention or pharmaceutically acceptable derivatives thereof and a sterile vehicle, optionally with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of formula (I) or pharmaceutically acceptable salts thereof may also be formulated in rectal compositions such as suppositories or retention enemas, e.g. containing conventional suppository bases such as cocoa butter or other glycerides.

The compounds of formula (I) or pharmaceutically acceptable salts thereof may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of formula (I) or pharmaceutically acceptable salts thereof, may be formulated as solutions for administration via a suitable metered or unitary dose device or alternatively as a powder mix with a suitable carrier for administration using a suitable delivery device. Thus compounds of formula (I) or pharmaceutically acceptable salts thereof may be formulated for oral, buccal, parenteral, topical (including ophthalmic and nasal), depot or rectal administration or in a form suitable for administration by inhalation or insufflation (either through the mouth or nose).

The compounds of formula (I) or pharmaceutically acceptable salts thereof may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

The composition may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, 1.0 to 500 mg or 1.0 to 200 mg and such unit doses may be administered more than once a day, for example two or three times a day.

Compounds of formula (I) or pharmaceutically acceptable salts thereof may be used in combination preparations. For example, the compounds of the invention may be used in combination with cyclosporin A, methotrexate, steroids, rapamycin, proinflammatory cytokine inhibitors, immunomodulators including biologicals or other therapeutically active compounds.

The subject invention also includes isotopically-labeled compounds, which are identical to those recited in formulas I and following, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹⁸F, ¹²³I and ¹²⁵I.

Compounds of the present invention and pharmaceutically acceptable salts of said compounds that contain the aforementioned isotopes and/or other isotopes of other atoms are within the scope of the present invention. Isotopically-labeled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula (I) and following of this invention can generally be prepared by carrying out the procedures disclosed in the Schemes and/or in the Examples below, by substituting a readily available isotopically labelled reagent for a non-isotopically labeled reagent.

In a further aspect, this invention provides processes for the preparation of a compound of formula (I). Compounds of formula (I) wherein R₁ and A are as defined for formula (I), R₂ and R₃ are hydrogen and R₄ is (CH₂)₁₋₃CO₂H may be prepared as described in Scheme 1. P is a protecting group and R represents an alkyl group such as ethyl.

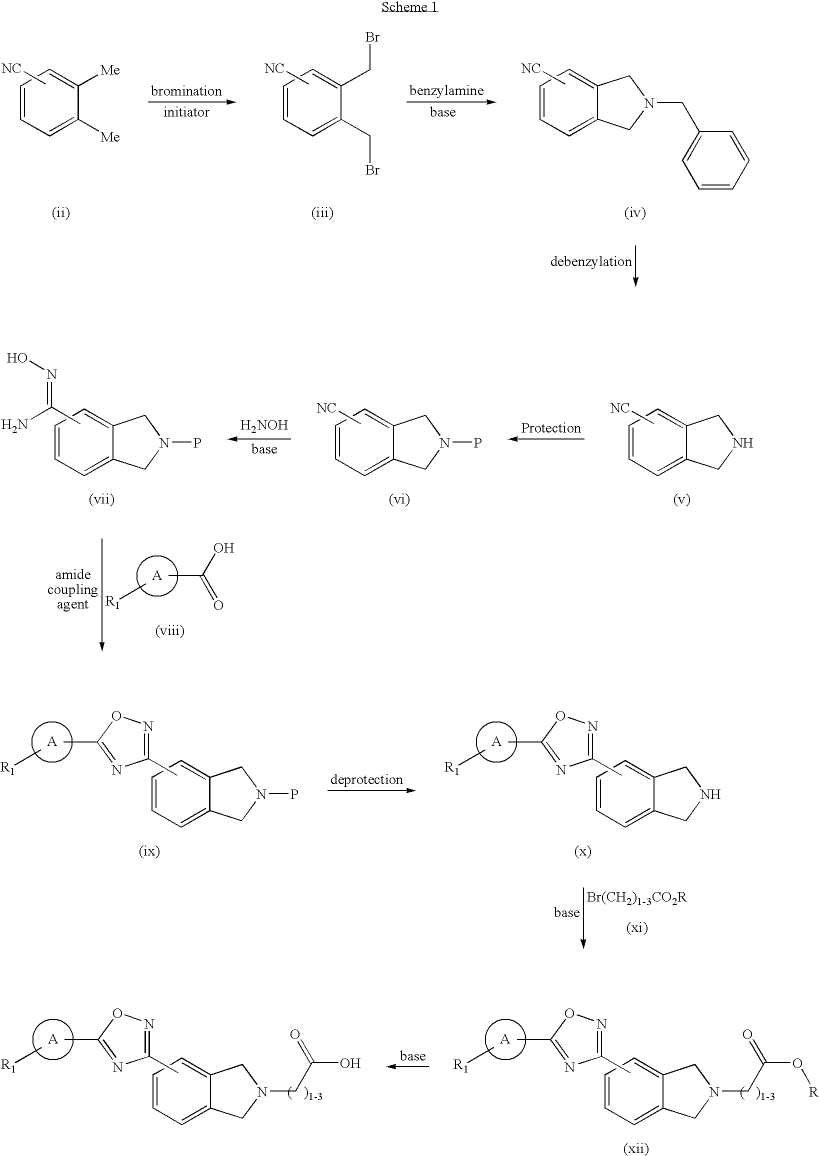

Compounds of formula (ii) (for example available from Fluorochem) may be converted into compounds of formula (iii) by reaction with a suitable brominating agent such as N-bromosuccinimide in the presence of a suitable radical initiator such as benzoyl peroxide. Compounds of formula (iii) may be converted into compounds of formula (iv) by reaction with benzylamine in the presence of a suitable base such as sodium carbonate. Compounds of formula (iv) may be converted into compounds of formula (v) by debenzylation, for example using 1-chloroethyl chloroformate. Compounds of formula (v) may be converted into compounds of formula (vi) wherein P represents a suitable protecting group such as tert-butoxycarbonyl, using a suitable protecting reagent such as bis(1,1-dimethylethyl) dicarbonate. Compounds of formula (vi) may be converted into compounds of formula (vii) by reaction with hydroxylamine in the presence of a suitable base such as sodium bicarbonate. Compounds of formula (vii) may be converted into compounds of formula (ix) by reaction with compounds of formula (viii) in the presence of a suitable amide coupling reagent such as N-(3-dimethylaminopropyl)-N′-ethylcarbodiimide hydrochloride in the presence of 1-hydroxybenzotriazole. Compounds of formula (ix) may be converted into compounds of formula (x) by deprotection; for example where P represents tert-butoxycarbonyl a suitable acid such as hydrogen chloride may be used. Compounds of formula (x) may be converted into compounds of formula (xii) by reaction with an alkylating agent of formula (xi) in the presence of a suitable base such as N,N-diisopropylethylamine. Compounds of formula (xii) may be converted into certain compounds of formula (I) by reaction with a suitable base such as sodium hydroxide. Compounds of formulae (viii) and (xi) are either commercially available, or are known compounds or may be prepared by conventional means.

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of compounds of the invention.

### ABBREVIATIONS

g—grams
mg—milligrams
ml—millilitres
ul—microlitres
MeCN—acetonitrile
MeOH—methanol
EtOH—ethanol
Et₂O—diethyl ether
EtOAc—ethyl acetate
DCM—dichloromethane
DIAD—diisopropyl azodicarboxylate
DME—1,2-bis(methyloxy)ethane
DMF—N,N-dimethylformamide
DMSO—dimethylsulphoxide
EDAC—N-(3-Dimethylaminopropyl)-N′-ethylcarbodiimide hydrochloride
EDC—N-(3-Dimethylaminopropyl)-N′-ethylcarbodiimide hydrochloride
EDCI—N-(3-Dimethylaminopropyl)-N′-ethylcarbodiimide hydrochloride
HOBT/HOBt—Hydroxybenzotriazole
IPA—isopropylalcohol
NCS—N-chlorosuccinimide
PyBOP—Benzotriazol-1-yl-oxytripyrrolidinophosphonium hexafluorophosphate
THF—tetrahydrofuran
dba—dibenzylidene acetone
RT—room temperature
° C.—degrees Celsius
M—Molar
H—proton
singlet
d—doublet
t—triplet
q—quartet
MHz—megahertz
MeOD—deuterated methanol
LCMS—Liquid Chromatography Mass Spectrometry
LC/MS—Liquid Chromatography Mass Spectrometry
MS—mass spectrometry
ES—Electrospray
MH⁺—mass ion+H⁺
MDAP—mass directed automated preparative liquid chromatography.
sat.—saturated

### General Chemistry Section

The methods described below are given for illustrative purposes, intermediates in the preparation of the examples may not necessarily have been prepared from the specific batches described.

### Description 1

### 2,3-Bis(bromomethyl)benzonitrile

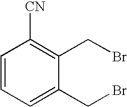

A mixture of 2,3-dimethylbenzonitrile (available from Fluorochem, 5 g, 38.1 mmol), N-bromosuccinimide (13.6 g, 76.2 mmol) and benzoyl peroxide (460 mg, 1.9 mmol) in carbon tetrachloride (270 ml) was stirred and heated at 80° C. for 20 hours then cooled and washed with 4×60 ml of water. The organic phase was dried (magnesium sulphate), evaporated and purified by flash chromatography eluting with ethyl acetate/iso-hexane (1:9) to give a yellow solid which was 58% pure by LC/MS.

¹H NMR (CDCl₃) δ: 4.63 (2H, s), 4.82 (2H, s), 7.43 (1H, t), 7.64 (2H, m).

### Description 2

### 2-(Phenylmethyl)-2,3-dihydro-1H-isoindole-4-carbonitrile

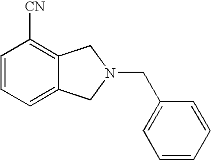

A mixture of 2,3-bis(bromomethyl)benzonitrile (D1) (47.9 g, 165.74 mmol), benzylamine (17.76 g, 165.74 mmol), and sodium carbonate (34.8 g, 414.35 mmol) in acetonitrile (500 ml) was stirred at 80° C. under argon for 2.75 hours then cooled and evaporated. The residue was dissolved in ethyl acetate (1 litre) and washed with 3×300 ml of potassium carbonate solution. The organic layer was dried (magnesium sulphate), evaporated and the residue dissolved in ethyl acetate (300 ml). The solution was filtered to remove some solid, the filtrate evaporated and the residue dissolved in acetone (280 ml) and acidified with 1M hydrogen chloride in ether. The precipitated solid was filtered off and partitioned between ethyl acetate and 2M sodium hydroxide. The organic phase was dried (magnesium sulphate), evaporated and purified by flash chromatography twice eluting first time with 6% 2M ammonia in methanol/dichloromethane and secondly using a gradient elution of 0-5% of 2M ammonia in methanol/dichloromethane to give 9.9 g of black oil.

LC/MS: [M+H]⁺235.0

### Description 3

### 2,3-Dihydro-1H-isoindole-4-carbonitrile

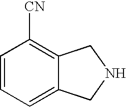

1-Chloroethyl chloroformate (11.88 g, 83.04 mmol) was added over ten minutes to a stirred mixture of 2-(phenylmethyl)-2,3-dihydro-1H-isoindole-4-carbonitrile (D2) (11.78 g, 50.34 mmol) and 4 A molecular sieves (5 g) in chlorobenzene (85 ml) and heated at 90° C. under argon for 2.75 hours. The reaction was cooled, methanol (200 ml) added and refluxed for one hour then left at room temperature for 3 days then evaporated to dryness. The residue was dissolved in ethyl acetate and extracted with 2M hydrochloric acid (500 ml and 2×300 ml) and the combined acid extracts basified with solid sodium hydroxide and extracted with ethyl acetate (3×200 ml). The combined organic extracts were dried with magnesium sulphate and evaporated to give 9.47 g of black oil.

LC/MS: [M+H]⁺145.1

### Description 4

### 1,1-Dimethylethyl 4-cyano-1,3-dihydro-2H-isoindole-2-carboxylate

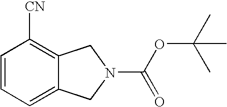

A mixture of 2,3-Dihydro-1H-isoindole-4-carbonitrile (D3) (9.47 g, 65.76 mmol), bis(1,1-dimethylethyl) dicarbonate (15.7 g, 72.3 mmol) and triethylamine (7.32 g, 72.3 mmol) in dichloromethane (200 ml) was stirred at room temperature for 5 hours then left overnight. The solution was washed with brine (4×120 ml), dried (magnesium sulphate), evaporated and purified by flash chromatography using a gradient elution from 10-20% of ethyl acetate in iso-hexane to give 5.52 g of white solid.

LC/MS: [M+H-56]⁺189.1

### Description 5

### 1,1-Dimethylethyl 4-[(hydroxyamino)(imino)methyl]-1,3-dihydro-2H-isoindole-2-carboxylate

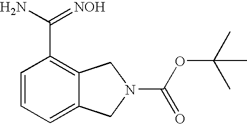

A mixture of 1,1-dimethylethyl 4-cyano-1,3-dihydro-2H-isoindole-2-carboxylate (D4) (1 g, 4.1 mmol), hydroxylamine hydrochloride (0.56 g, 8.2 mmol) and sodium bicarbonate (1.72 g, 20.5 mmol) in ethanol (30 ml) was stirred and heated at 50° C. for 6.5 hours then left overnight at room temperature. The mixture was evaporated to dryness and partitioned between water (100 ml) and ethyl acetate (100 ml) and the organic phase washed with water (3×100 ml), dried (magnesium sulphate) and evaporated to give 1.33 g of a viscous yellow oil.

LC/MS: [M+H]⁺278.0

### Description 6

### 1,1-Dimethylethyl 4-(5-{3-cyano-4-[(1-methylethyl)oxy]phenyl}-1,2,4-oxadiazol-3-yl)-1,3-dihydro-2H-isoindole-2-carboxylate

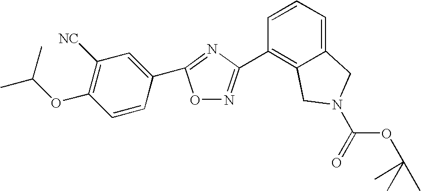

A mixture of 1,1-dimethylethyl 4-[(hydroxyamino)(imino)methyl]-1,3-dihydro-2H-isoindole-2-carboxylate (D5) (1.33 g, 4.8 mmols), 3-cyano-4-[(1-methylethyl)oxy]benzoic acid (available from AK Scientific Product Catalog, 984 mg, 4.8 mmol), 1-hydroxybenzotriazole (HOBt, 802 mg, 5.28 mmol) and N-(3-dimethylaminopropyl)-N′-ethylcarbodiimide hydrochloride (EDC, 1.01 g, 5.28 mmol) was stirred and heated at 80° C. for 24 hours. The solution was cooled, diluted with ethyl acetate/water and the organic phase washed with 5×100 ml of water, dried (magnesium sulphate), evaporated and purified by flash chromatography using a gradient elution from 10-20% ethyl acetate in iso-hexane. The product containing fractions were diluted with ethyl acetate (300 ml), washed with saturated sodium bicarbonate solution, dried (magnesium sulphate), evaporated and the residue triturated with 1:1 ether/hexane to give 230 mg of white solid.

LC/MS: [2M+H]⁺893.5

The following compound was prepared in a similar manner to Description 6 by reaction of the appropriate amidoxime with the appropriate acid using EDC and HOBt.

| D7 | Name | LC/MS |
|---|---|---|
| 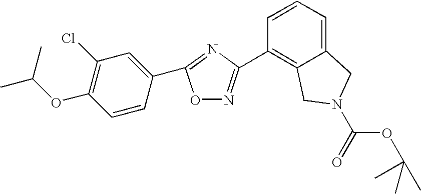 | 1,1-Dimethylethyl 4-(5-{3- chloro-4-[(1- methylethyl)oxy]phenyl}- 1,2,4-oxadiazol-3-yl)-1,3- dihydro-2H-isoindole-2- carboxylate | [M + H − 56]⁺ 399.9 |

### Description 8

### Ethyl 3-[4-(5-{3-chloro-4-[(1-methylethyl)oxy]phenyl}-1,2,4-oxadiazol-3-yl)-1,3-dihydro-2H-isoindol-2-yl]propanoate

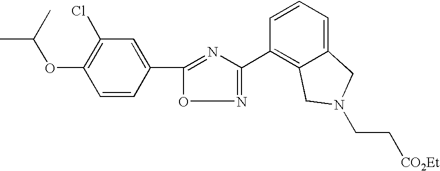

A solution of 4-(5-{3-chloro-4-[(1-methylethyl)oxy]phenyl}-1,2,4-oxadiazol-3-yl)-2,3-dihydro-1H-isoindole (Example 2) (103 mg, 0.29 mmol), N,N-diisopropylethylamine (75 mg, 0.58 mmol) and ethyl 3-bromopropionate (79 mg, 0.44 mmol) in acetonitrile (2 ml) was stirred and heated at 80° C. under argon for 2 hours then left at room temperature overnight. The resulting solution was diluted with ethyl acetate (40 ml) and washed with saturated sodium bicarbonate (3×30 ml) and water (30 ml) then dried (magnesium sulphate) and evaporated to give 123 mg of brown oil.

LC/MS: [M+H]⁺455.9

The following compounds were prepared in a similar manner by alkylation of Example 2 (D9) or Example 1 (D10) with ethyl 4-bromobutanoate.

| Description | Name | LC/MS |
|---|---|---|
| 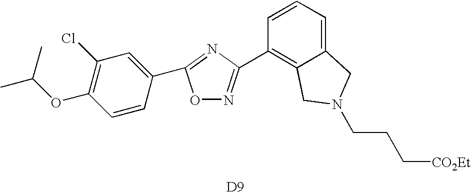 | Ethyl 4-[4-(5-{3-chloro-4- [(1- methylethyl)oxy]phenyl}- 1,2,4-oxadiazol-3-yl)-1,3- dihydro-2H-isoindo1-2- yl]butanoate | [M + H]⁺ 469.9 |
| 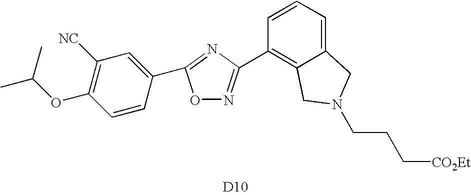 | Ethyl 4-[4-(5-{3-cyano-4- [(1- methylethyl)oxy]phenyl}- 1,2,4-oxadiazol-3-yl)-1,3- dihydro-2H-isoindol-2- yl]butanoate | [M + H]⁺ 461.0 |

### EXAMPLE 1

### 5-[3-(2,3-Dihydro-1H-isoindol-4-yl)-1,2,4-oxadiazol-5-yl]-2-[(1-methylethyl)oxy]benzonitrile hydrochloride

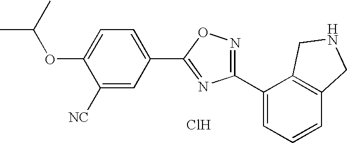

1,1-Dimethylethyl 4-(5-{3-cyano-4-[(1-methylethyl)oxy]phenyl}-1,2,4-oxadiazol-3-yl)-1,3-dihydro-2H-isoindole-2-carboxylate (D6, 0.28 g, 0.627 mmol) was dissolved in 1,4-dioxane (3 ml) (heating required) and 4M hydrogen chloride in 1,4-dioxane (4.70 ml, 18.81 mmol) added. The mixture was left at room temperature for one during which time solid separated. The solvent was evaporated, the residue triturated with ether and the solid filtered off and dried to give 211 mgs of the title compound as an off-white solid.

LC/MS: [M+H]⁺347.0

¹H NMR (MeOD) δ: 1.46 (6H, d), 4.73 (2H, s), 4.95 (1H, m), 5.02 (2H, s), 7.46 (1H, d), 7.64 (2H, m), 8.26 (1H, dd), 8.44 (1H, dd), 8.48 (1H, d).

### EXAMPLE 2

### 4-(5-{3-Chloro-4-[(1-methylethyl)oxy]phenyl}-1,2,4-oxadiazol-3-yl)-2,3-dihydro-1H-isoindole

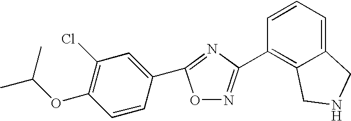

1,1-dimethylethyl 4-(5-{3-chloro-4-[(1-methylethyl)oxy]phenyl}-1,2,4-oxadiazol-3-yl)-1,3-dihydro-2H-isoindole-2-carboxylate (D7, 314 mg, 0.69 mmol) was dissolved in dichloromethane (2 ml) and TFA (2 ml) and the mixture left at room temperature for 30 minutes. The mixture was evaporated, azeotroped with toluene (4×10 ml) and the residue dissolved in ethyl acetate (50 ml) and 2M sodium hydroxide (30 ml). The organic phase was dried (magnesium sulphate) and evaporated to give the title compound (236 mg) as a solid.

LC/MS: [M+H]⁺356.1

¹H NMR (CDCl₃) δ: 1.45 (6H, d), 4.34 (2H, s), 4.65 (1H, m), 4.72 (2H, s), 7.06 (1H, d), 7.39 (2H, m), 8.07 (2H, m), 8.24 (1H, d).

### EXAMPLE 3

### 3-[4-(5-{3-Chloro-4-[(1-methylethyl)oxy]phenyl}-1,2,4-oxadiazol-3-yl)-1,3-dihydro-2H-isoindol-2-yl]propanoic acid

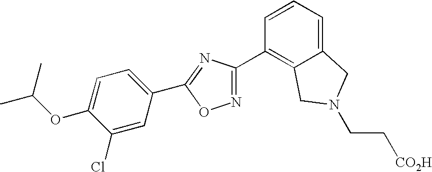

A solution of ethyl 3-[4-(5-{3-chloro-4-[(1-methylethyl)oxy]phenyl}-1,2,4-oxadiazol-3-yl)-1,3-dihydro-2H-isoindol-2-yl]propanoate (D8) (123 mg, 0.27 mmol) in ethanol (5 ml) and 2M sodium hydroxide solution (2 ml) was stirred at room temperature for 30 minutes. The mixture was evaporated to dryness and the residue dissolved in water (20 ml), treated with acetic acid (0.2 ml) and extracted with ethyl acetate (50 ml). The aqueous phase was evaporated to dryness then redissolved in water (60 ml), acidified with acetic acid (0.2 ml) and extracted with ethyl acetate (60 ml). The organic phase was dried (magnesium sulphate), evaporated, and the residue azeotroped with toluene (4×10 ml) and triturated with ether to give the title compound (38 mg) as a brown solid. LC/MS [M+H]⁺428.0

¹H NMR (CDCl₃) δ: 1.44 (6H, d), 2.67 (2H, t), 3.22 (2H, t), 4.23 (2H, s), 4.55 (2H, s), 4.69 (1H, m), 7.01 (1H, d), 7.39 (2H, m), 8.01 (1H, d), 8.10 (1H, m), 8.18 (1H, d).

The following compounds were prepared in a similar manner to Example 3 by hydrolysis of the appropriate ester (D9 for Example 4 and D10 for Example 5).

| Example | Name | LC/MS, NMR |
|---|---|---|
| 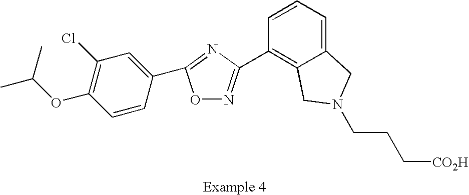 | 4-[4-(5-{3-Chloro-4-[(1- methylethyl)oxy]phenyl}- 1,2,4-oxadiazol-3-yl)-1,3- dihydro-2H-isoindol-2- yl]butanoic acid | [M + H]⁺ 441.9, 443.9 ¹H NMR (MeOD) δ: 1.42 (6H, d), 2.13 (2H, m), 2.54 (2H, t), 3.58 (2H, t), 5.15 (2H, s), 4.55 (2H, s), 7.32 (1H, d), 7.65 (2H, m), 8.14 (1H, dd),8.24 (2H, m). |
| 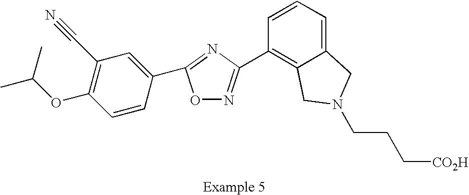 | 4-[4-(5-{3-cyano-4-[(1- methylethyl)oxy]phenyl}- 1,2,4-oxadiazol-3-yl)-1,3- dihydro-2H-isoindol-2- yl]butanoic acid | [M + H]⁺ 433.1 ¹H NMR (DMSO) δ: 1.38 (6H, d), 1.78 (2H, m), 2.32 (2H, t), 2.75 (2H, t), 3.96 (2H, s), 4.22 (2H, s), 4.98 (1H, m), 7.475 (2H, m), 7.55 (1H, d), 7.97 (1H, d), 8.41 (1H, d), 8.51 (1H, s) |

### Membrane Preparation for S1P1 GTPγS Assay

For membrane preparations all steps were performed at 4° C. Rat hepatoma cells stably expressing the human S1P1 receptor or Rat Basophilic Leukaemia cells (RBL) stably expressing human S1P3 receptor were grown to 80% confluency before being harvested into 10 ml Phospho-Buffered Saline (PBS) and centrifuged at 1200 rpm for 5 minutes. After removal of the supernatant, the pellet was re-suspended and cells were homogenised within a glass Waring blender for 2 bursts of 15 secs in 200 mls of buffer (50 mM HEPES, 1 mM leupeptin, 25 μg/ml bacitracin, 1 mM EDTA, 1 mM PMSF, 2 μM pepstatin A). The blender was plunged into ice for 5 mins after the first burst and 10-40 mins after the final burst to allow foam to dissipate. The material was then spun at 500 g for 20 mins and the supernatant spun for 36 mins at 48,000 g. The pellet was resuspended in the same buffer as above but without PMSF and pepstatin A. The material was then forced through a 0.6 mm needle, made up to the required volume, (usually ×4 the volume of the original cell pellet), aliquoted and stored frozen at −80° C.

### Alternative Membrane Preparation for S1P1 GTPγS Assay

All steps were performed at 4° C. Cells were homogenised within a glass Waring blender for 2 bursts of 15 secs in 200 mls of buffer (50 mM HEPES, 1 mM leupeptin, 25 μg/ml bacitracin, 1 mM EDTA, 1 mM PMSF, 2 μM pepstatin A). The blender was plunged into ice for 5 mins after the first burst and 10-40 mins after the final burst to allow foam to dissipate. The material was then spun at 500 g for 20 mins and the supernatant spun for 36 mins at 48,000 g. The pellet was resuspended in the same buffer as above but without PMSF and pepstatin A. The material was then forced through a 0.6 mm needle, made up to the required volume, (usually ×4 the volume of the original cell pellet), aliquoted and stored frozen at −80° C.

### S1P1 GTPγS Assay

Human S1P1 rat hepatoma membranes (1.5 μg/well) were adhered to a wheatgerm agglutinin (WGA)-coated scintillation proximity assay (SPA) beads (0.125 mg/well) in assay buffer (HEPES 20 mM, MgCl₂ 10 mM, NaCl 100 mM and pH adjusted to 7.4 using KOH 5M, GDP 10 μM FAC (final assay concentration) and saponin 90 μg/ml FAC was also added).

After 30 minutes pre-coupling on ice the bead and membrane suspension was dispensed into a white Greiner polypropylene LV384-well plate (5 μl/well), containing 0.1 μl of the compound. 5 μl/well [³⁵S]-GTPγS (0.5 nM final radioligand conc) made up in assay buffer was then added to agonist plates. The final assay cocktail (10.1 μl) was then centrifuged at 1000 rpm for 5 minutes then read immediately on a Viewlux reader.

All test compounds were dissolved in DMSO at a concentration of 10 mM and were prepared in 100% DMSO using a 1 in 4 dilution step to provide 11 point dose response curves. The dilutions were transferred to the assay plates ensuring that the DMSO concentration was constant across the plate for all assays.

All data was normalized to the mean of 16 high and 16 low control wells on each plate. A four parameter curve fit was then applied.

Alternative method for S1P1 GTPγS Assay

S₁P₁ expressing RH7777 membranes (1.5 μg/well) membranes (1.5 μg/well) were homogenised by passing through a 23G needle. These were then adhered to WGA-coated SPA beads (0.125 mg/well) in assay buffer (HEPES 20 mM, MgCl₂ 10 mM, NaCl 100 mM and pH adjusted to 7.4 using KOH 5M). GDP 10 μM FAC and saponin 90 μg/ml FAC were also added

After 30 minutes precoupling on ice, the bead and membrane suspension was dispensed into white Greiner polypropylene LV 384-well plates (5 μl/well), containing 0.1 μl of compound. 5 μl/well [³⁵S]-GTPγS (0.5 nM for S₁P₁ or 0.3 nM for S₁P₃ final radioligand concentration) made in assay buffer was then added to the plates. The final assay cocktail (10.1 μl) was then sealed, spun on a centrifuge, then read immediately on a Viewlux instrument.

Examples 1 to 5 had a pEC50>6. Examples 1 and 2 had a pEC50 of >8.

### S1P3

S1P3 membranes from rat basophilic leukaemia cells (RBL-2H3) (1.5 μg/well) were adhered to WGA-coated SPA beads (0.125 mg/well) in assay buffer (HEPES 20 mM, MgCl₂ 3 mM, NaCl 100 mM and pH adjusted to 7.4 using KOH 5M), GDP 10 μM FAC and saponin 90 μg/ml FAC was also added).

After 30 minutes pre-coupling on ice the bead and membrane suspension was dispensed into a white Greiner polypropylene LV384-well plate (5 μl/well), containing 0.1 μl of the compound. 5 μl/well [³⁵S]-GTPγS (0.5 nM final radioligand conc) made up in assay buffer was then added to agonist plates. The final assay cocktail (10.1 μl) was centrifuged at 1000 rpm for 5 minutes then read immediately on a Viewlux reader.

All test compounds were dissolved in DMSO at a concentration of 10 mM and were prepared in 100% DMSO using a 1 in 4 dilution step to provide 11 point dose response curves. The dilutions were transferred to the assay plates ensuring that the DMSO concentration was constant across the plate for all assays.

All data was normalized to the mean of 16 high and 16 low control wells on each plate. A four parameter curve fit was then applied.

### Alternative Method for S1P3 GTPγS Assay

S₁P₃ expressing RBL membranes (1.5 μg/well) were homogenised by passing through a 23G needle. These were then adhered to WGA-coated SPA beads (0.125 mg/well) in assay buffer (HEPES 20 mM, MgCl₂ 10 mM, NaCl 100 mM and pH adjusted to 7.4 using KOH 5M). GDP 10 μM FAC and saponin 90 m/ml FAC were also added

After 30 minutes precoupling on ice, the bead and membrane suspension was dispensed into white Greiner polypropylene LV 384-well plates (5 μl/well), containing 0.1 μl of compound. 5 μl/well [³⁵S]-GTPγS (0.5 nM for S₁P₁ or 0.3 nM for S₁P₃ final radioligand concentration) made in assay buffer was then added to the plates. The final assay cocktail (10.1 μl) was then sealed, spun on a centrifuge, then read immediately on a Viewlux instrument.

Examples 1 to 5 had a pEC50<6. Examples 1, 2 and 5 had a pEC50<5.

1. A compound of formula (I) or a pharmaceutically acceptable salt thereof: 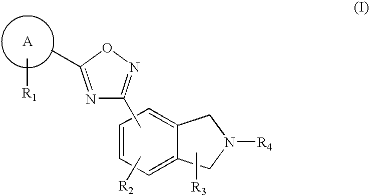
A is phenyl or a 5 or 6-membered heteroaryl ring;
R₁ is up to two substituents independently selected from halogen, C₍₁₋₃₎alkoxy, C₍₁₋₃₎fluoroalkyl, cyano, optionally substituted phenyl, C₍₁₋₃₎fluoroalkoxy, C₍₁₋₆₎alkyl and C₍₃₋₆₎cycloalkyl;
R₂ is hydrogen, halogen or C₁₋₄alkyl;
R₃ is hydrogen or C₁₋₃alkyl;
R₄ is hydrogen or (CH₂)₁₋₃CO₂H;
when R₂ or R₃ is C₁₋₃alkyl it may be optionally interrupted by O.

2. A compound of formula (I) or a pharmaceutically acceptable salt thereof, wherein:
A is phenyl;
R₁ is up to two substituents independently selected from chloro, isopropoxy, and cyano;
R₂ is hydrogen;
R₃ is hydrogen;
R₄ is hydrogen or (CH₂)₁₋₃CO₂H.

3. A compound selected from:
5-[3-(2,3-Dihydro-1H-isoindol-4-yl)-1,2,4-oxadiazol-5-yl]-2-[(1-methylethyl)oxy]benzonitrile
4-(5-{3-Chloro-4-[(1-methylethyl)oxy]phenyl}-1,2,4-oxadiazol-3-yl)-2,3-dihydro-1H-isoindole
3-[4-(5-{3-Chloro-4-[(1-methylethyl)oxy]phenyl}-1,2,4-oxadiazol-3-yl)-1,3-dihydro-2H-isoindol-2-yl]propanoic acid
4-[4-(5-{3-Chloro-4-[(1-methylethyl)oxy]phenyl}-1,2,4-oxadiazol-3-yl)-1,3-dihydro-2H-isoindol-2-yl]butanoic acid
4-[4-(5-{3-cyano-4-[(1-methylethyl)oxy]phenyl}-1,2,4-oxadiazol-3-yl)-1,3-dihydro-2H-isoindol-2-yl]butanoic acid
and pharmaceutically acceptable salts thereof.

4. Use of a compound according to any one of claims 1 to 3 for the treatment of conditions or disorders mediated by S1P1 receptors.

5. Use according to claim 4, wherein the condition or disorder is multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

6. Use according to claim 4, wherein the condition is lupus erythematosis.

7. Use of a compound according to any one of claims 1 to 3 to manufacture a medicament for use in the treatment of conditions or disorders mediated by S1P1 receptors.

8. Use according to claim 7, wherein the condition or disorder multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

9. Use according to claim 7, wherein the condition is lupus erythematosis.

10. A pharmaceutical composition comprising a compound according to any one of claims 1 to 3.

11. A method of treatment for conditions or disorders in mammals including humans which can be mediated via the S1P1 receptors which comprises administering to the sufferer a therapeutically safe and effective amount of a compound of formula (I) or a pharmaceutically acceptable salt thereof.

12. A method of treatment according to claim 11, wherein the condition is lupus erythematosis.
